(19) United States
(12) Patent Application Publication
(10) Pub. No.: US 2014/0322344 A1
(43) Pub. Date: Oct. 30, 2014

(54) VACCINE PREPARATION FOR CANCER TREATMENT

(71) Applicants: Hiroshi Shiku, Tsu-shi (JP); Naozumi Harada, Tokyo (JP); Daisuke Muraoka, Tokyo (JP); Kazunari Akiyoshi, Kyoto-shi (JP)

(72) Inventors: Hiroshi Shiku, Tsu-shi (JP); Naozumi Harada, Tokyo (JP); Daisuke Muraoka, Tokyo (JP); Kazunari Akiyoshi, Kyoto-shi (JP)

(73) Assignees: National University Corporation Tokyo Medical and Dental University, Tokyo (JP); MIE University, Tsu-shi (JP)

(21) Appl. No.: 14/241,321

(22) PCT Filed: Aug. 30, 2012
(86) PCT No.: PCT/JP2012/071979

§ 371 Date: May 29, 2014

(30) Foreign Application Priority Data
Aug. 31, 2011 (JP) 2011-189234

(51) Int. Cl. A61K 39/385; A61K 39/39

(52) U.S. Cl. CPC A61K 39/385; A61K 39/39; A61K 2039/6087
USPC 424/499; 424/193.1; 424/196.11; 530/322

(57) ABSTRACT

A vaccine preparation for treating cancer includes a complex of a hydrophobized polysaccharide and at least one synthetic long peptide derived from a tumor-specific antigenic protein and/or a pathogen-derived antigenic protein. The at least one synthetic long peptide contains at least one CD8+ cytotoxic T-cell recognition epitope and at least one CD4+ helper T-cell recognition epitope. The complex is simultaneously administered to the patient with at least one immunopotentiating agent.

### TECHNICAL FIELD

The present invention relates to a cancer therapy, particularly a vaccine for cancer treatment. More specifically, it relates to a vaccine preparation for cancer treatment comprising a synthetic long peptide as a vaccine antigen, a hydrophobized polysaccharide as an antigen delivery system, and an immunopotentiating agent which stimulates antigen-presenting cells.

### BACKGROUND ART

Results of many years of research concerning immunological responses to cancers have made clear the importance of cellular immunity in tumor rejection of a cancer host. In particular, it has become clear that CD8-positive cytotoxic T cells (hereinafter, killer T cells) are effector cells having the function of directly disrupting tumors, CD4-positive helper T cells (hereinafter, helper T cells) are important regulatory cells which enhance the activities of killer T cells and antigen-presenting cells, and antigen-presenting cells, of which dendritic cells play a leading role, present antigen to the T cells to stimulate them and to activate T cells through various co-stimulatory molecules, cytokines and the like; as described below, the roles and positionings of each cell responsible for cellular immunological responses to tumors have been established (Non Patent Document 1).

After protein derived from a tumor cell and protein administered as a vaccine antigen are internalized in an antigen-presenting cell such as a dendritic cell, they are cleaved into peptides of various lengths by proteases in the cell. Among the resulting peptides, peptides having 8-10 amino acids are loaded onto major histocompatibility complex (MHC) class I molecules as antigenic epitope peptides and can be presented on the cell surface. Killer T cells specifically recognize these MHC class I/antigenic peptide complexes using T cell receptors (TCR) and are activated. The activated killer T cells detect MHC class I/antigenic peptide complexes also present on tumor cells and disrupt the tumor cells using effector molecules such as granzyme and perforin.

The activity of helper T cells is very important for sufficiently activating killer T cells (Non Patent Document 2). Antigenic protein that has been internalized in an antigen-presenting cell such as a dendritic cell is cleaved into various lengths by the proteases in the cell; antigenic peptides thereof having 15-20 amino acids form complexes with MHC class II molecules and are presented on the antigen-presenting cell. Helper T cells specifically recognize these and are activated. The activated helper T cells enhance the proliferation, survival and function of killer T cells through the secretion of cytokines such as interferon (IFN)-γ and interleukin (IL)-2. In addition, helper T cells have the function of activating dendritic cells through the CD40 ligand/CD40 pathway; dendritic cells activated by helper T cells increase the stimulus capability for killer T cells (Non Patent Document 3). It is well known in the art that helper T cells also have the function of enhancing the antigen-specific IgG antibody production of B cells.

Thus, killer T cells are activated by antigen-presenting cells such as dendritic cells in an antigen-specific manner, and helper T cells behave as important enhancers that further enhance the activities of both killer T cells and dendritic cells. All of these three types of immunocytes are essential for the effective activation of cellular immunity to tumors. The inventors have reported that a vaccine, which stimulates only killer T cells but does not activate helper T cells, induces only inferior killer T cells and does not exhibit a therapeutic effect (Non Patent Document 4); thus an important task in the development of vaccines for cancer treatment is how to cause these three types of immunocytes to activate and effectively function.

In the past, the inventors prepared a polyvalent vaccine for cancer treatment, wherein the antigen was a recombinant full-length protein of a tumor antigenic protein, with the goal of co-activating killer T cells and helper T cells. A variety of antigenic peptides, which killer T cells and helper T cells respectively recognize, were contained in the full-length protein, and were expected to co-activate both T cells. However, although exogenous (extracellular) antigenic protein readily fostered the activation of helper T cells via the MHC class II pathway, activation of killer T cells via the MHC class I pathway was scarcely fostered. This resulted from the mechanism of internalizing and processing of exogenous antigenic protein in antigen-presenting cells (Non Patent Document 5).

Thus, in Japan and other countries, many clinical trials have been conducted using short-chain peptides as vaccines, in which epitope peptides having 8-10 residues that mainly killer T cells recognize have been chemically synthesized. Since short-chain peptides directly bind to MHC molecules on the cell surface without being internalized and processed in antigen-presenting cells, presentation to the T cell is likely to occur. In addition, production and purification of recombinant full-length protein requires the use of E. coli, mammalian cells or the like, and the construction and quality control of a manufacturing system require a great deal of time and effort. In contrast thereto, short-chain peptides have the advantage that they can be manufactured by chemical synthesis, and can be more easily manufactured than recombinant proteins.

However, some important issues have been identified with respect to vaccines for cancer treatment that comprise short-chain peptides. Since many short-chain peptide vaccines contain only recognition epitope peptides of killer T-cells, they are monovalent vaccines that do not involve activation of helper T cells, and the quality of the induced cellular immunity and the therapeutic effects are insufficient (Non Patent Document 4). If the vaccine is prepared by synthesizing recognition epitopes of each of killer T cells and helper T cells as short-chain peptides and it is comprised of a mixture thereof, it is possible to achieve therapeutic effects with induction of high quality killer T cells by co-activation of killer T cells and helper T cells. However, in this case, since the recognition epitope peptides of the killer T cells and the helper T cells are administered as discrete components, different dendritic cells present the respective peptides, and there is a high likelihood that it will not lead to interaction of the killer T cells and the helper T cells (Non Patent Document 3).

In addition, a problem also has been identified with respect to the short-chain peptides directly binding to the MHC molecules on the cell surface without being internalized and processed in antigen-presenting cells (Non Patent Document 6). That is, exogenous antigenic proteins are phagocytized by professional antigen-presenting cells, which have co-stimulatory molecules similar to dendritic cells and macrophages, and are processed in the cell; although antigens are presented to the T cells at an adequate concentration in a manner that involves co-stimulation, because the short-chain peptides directly bind to the MHC molecules on the cell surface without undergoing such processing, the peptides of interest are presented in a large amount in an unsuitable manner also in ordinary somatic cells which generally have a limited ability to internalize (phagocytize) and process antigen, and do not express co-stimulatory molecules, and there is a possibility of causing immunological tolerance.

In view of the problems of vaccines that comprise such short-chain peptides, in recent years the usefulness has been proposed of long-chain synthetic peptides (long peptides) which incorporate each of the advantages of full-length protein capable of co-activating killer T cells and helper T cells and inexpensive short-chain peptides that are easily manufactured (Non Patent Document 6). A long peptide is typically a polypeptide having dozens of residues which comprise at least one recognition epitope peptide of a killer T-cell and at least one recognition epitope peptide of a helper T-cell. Co-activation of killer T cells and helper T cells by antigen-presenting cells that have internalized the long peptide is expected. In addition, since chemical synthesis methods can be used, vaccines having a long peptide as the antigen have the advantage that their manufacture is relatively easy, similar to short-chain peptides.

Furthermore, unlike short-chain peptides, long peptides cannot directly bind to an MHC molecule as is. Because a long peptide undergoes processing in cells that internalize them such as dendritic cells, recognition epitope peptides of killer T cells and helper T cells contained in the long peptide form complexes with MHC molecules and are presented to the T cells in an appropriate concentration and manner. Additionally, since long peptides do not function as a vaccine antigen in somatic cells that generally lack the ability to internalize and process antigens, inappropriate antigen presentation to T cells does not occur.

From the above, vaccines for cancer treatment, wherein a synthetic long peptide serves as the vaccine antigen, are expected to be capable of being excellent cancer medicines that can be manufactured at a relatively low cost while also being capable of co-activating killer T cells and helper T cells. However, even in vaccines used for cancer treatment wherein a synthetic long peptide serves as the vaccine antigen, since a sufficient number of T cells cannot be activated in case it is administered alone, efficient activation of antigen-presenting cells such as dendritic cells is required to maximize the effectiveness of the vaccine.

Immunopotentiating agents (adjuvants) have been conventionally used in order to enhance the immunogenicity of vaccines. A variety of substances derived from bacteria and viruses, for example, nucleic acids (DNA and RNA), proteins, lipopolysaccharides and the like, have been used as immunopotentiating agents; in recent research it has become clear that these substances bind to Toll-like receptors on cell surfaces and on intracellular sites that are required for activation of dendritic cells to increase the expression of co-stimulatory molecules (CD80 and CD86) and MHC molecules, and remarkably improve the stimulatory activity for the T cells through the production of interferons, cytokines and the like. In addition to agonists to Toll-like receptors, it has been reported that chemotherapeutic agents such as taxane-based compounds also have the effect of activating dendritic cells (Non Patent Document 7), and that signal transduction inhibitors, which act to prevent dendritic cells from acquiring immunosuppressive activity, can also be effective to enhance vaccine effects (Non Patent Document 8); thus there is a potential that such substances can be utilized as immunopotentiating agents.

Additionally, the inventors have conducted research on vaccine antigen delivery systems that facilitate killer T cell activation through the MHC class I pathway of exogenous antigenic proteins in antigen-presenting cells such as dendritic cells and the like (called cross-presentation or cross-priming) and have found such functions in hydrophobized polysaccharides (Patent Document 1). For example, a vaccine preparation, in which an HER2 full-length protein, which is a tumor antigen protein, was complexed with cholesteryl pullulan (abbr.: CHP) or cholesteryl mannan (abbr.: CHM), which are types of hydrophobized polysaccharides, exhibited superior anti-tumor effects when it efficiently activated not only helper T cells but also killer T cells in the case of administration to human dendritic cells in vitro, and also when administered to a tumor-bearing mouse model, as compared to HER2 full-length protein that was administered alone (Non Patent Document 9). Similar results have also been reproduced in the case of using NY-ESO-1 full-length protein, which is another tumor antigenic protein (Non Patent Document 10). However, the usefulness in low molecular weight, long peptides is uncertain.

In addition, it is becoming clear that the size and surface charge of the vaccine antigen delivery system are directly associated with the vaccine performance and are thus important. In liposomes, which are currently a widely-used vaccine antigen delivery system, there are reports that the larger the size is or the higher the charge is, the better the T cell activation and the antibody production inducement by the vaccine are (Non Patent Documents 14-16). However, in hydrophobized polysaccharides serving as a vaccine antigen delivery system, the conditions thereof are uncertain.

### CITATION LIST

### Patent Documents

Patent Document 1: Japanese Patent No. 4033497

Patent Document 2: Japanese Laid-open Patent Publication No. S61-69801

Patent Document 3: Japanese Laid-open Patent Publication No. H3-292301

Patent Document 4: Japanese Laid-open Patent Publication No. H7-97333

### Non Patent Documents

Non Patent Document 1: Ribas, A. et al. J. Clin. Oncol. 2003; 21(12): 2415-2432.

Non Patent Document 2: Shiku, H. Int. J. Hematol. 2003; 77(5): 435-8.

Non Patent Document 3: Behrens, G. et al. Immunol. Cell Biol. 2004; 82(1): 84-90.

Non Patent Document 4: Muraoka, D., et al. J. Immunol. 2010; 185(6): 3768-76.

Non Patent Document 5: Shen L. & Rock K. L. Curr. Opin. Immunol. 2006; 18(1): 85-91.

Non Patent Document 6: Melief, C. J. M., & van der Burg, S. H. Nature Rev. Cancer, 2008; 8(5): 351-360.

Non Patent Document 7: Byrd-Leifer, C. A., et al. Eur. J. Immunol. 2001; 31(8): 2448-57.

Non Patent Document 8: Kong, L. Y., et al. Clin. Cancer Res. 2008; 14(18): 5759-68.

Non Patent Document 9: Gu, X. G., et al. Cancer Res., 1998; 58(15): 3385-90.

Non Patent Document 10: Hasegawa, K., et al. Clin. Cancer Res., 2006; 12(6): 1921-27.

Non Patent Document 11: Akiyoshi, K., et al. Macromolecules., 1993; 26(12): 3062-68.

Non Patent Document 12: Akiyoshi, K., et al. J. Proc. Japan. Acad., 1995; 71(71B): 15.

Non Patent Document 13: Nishikawa, T., et al. Macromolecules. 1994; 27(26): 7654-59.

Non Patent Document 14: Brewer, J. M., et al. J. Immunol. 1998; 161: 4000-7.

Non Patent Document 15: Henriksen-Lacey, M., et al. J. Controlled Release. 2011; 154(2): 131-7.

Non Patent Document 16: Nakanishi, T., et al. J. Controlled Release. 1999; 61: 233-40.

### SUMMARY OF THE INVENTION

### Problem(s) to be Solved by the Invention, and Means for Solving the Problem (s)

The inventors prepared long peptides that simultaneously contain recognition epitopes of killer T cells and helper T cells, which epitopes are derived from a tumor-specific antigenic protein and/or a pathogen-derived antigenic protein, and researched methods for maximizing therapeutic effects on cancers. As a result, after extensive research, the inventors found that, only after combining a complex of a synthetic long peptide and a hydrophobized polysaccharide CHP that is an antigen delivery system with an immunopotentiating agent, remarkable activation of killer T cells and helper T cells could be induced in a mouse model, and this basically led to completing the present invention.

Thus, a vaccine preparation for cancer treatment related to the present invention for solving the problems comprises a drug that includes a complex of a hydrophobized polysaccharide and one or more synthetic long peptides derived from a tumor-specific antigenic protein and/or a pathogen-derived antigenic protein and that simultaneously contain(s) a CD8 positive, cytotoxic T-cell recognition epitope and a CD4 positive, helper T-cell recognition epitope, and one or more immunopotentiating agents.

At this time, the synthetic long peptide is preferably a polypeptide including 23-120 amino acids that include at least two or more T-cell recognition epitopes. Also, the synthetic long peptide is preferably a polypeptide including 23-80 amino acids that include at least two or morel-cell recognition epitopes. Also, the synthetic long peptide is preferably a polypeptide including 23-40 amino acids that include at least two or more T-cell recognition epitopes.

Also, the CD8 positive, cytotoxic T-cell recognition epitope is preferably a part of the amino acid sequence of the tumor-specific antigenic protein.

Also, the CD4 positive, helper T-cell recognition epitope is preferably a part of the amino acid sequence of the tumor-specific antigenic protein.

At this time, the tumor-specific antigenic protein is preferably selected from the MAGE family or NY-ESO-1, the SPANX family, HER2, WT1, hTERT, RHAMM and survivin.

Also, the CD8-positive helper T-cell recognition epitope is preferably a part of the amino acid sequence of the pathogen-derived protein.

Also, the CD4 positive, helper T-cell recognition epitope is preferably a part of the amino acid sequence of the pathogen-derived protein.

At this time, the pathogen-derived antigenic protein is preferably selected from hepatitis B virus, hepatitis C virus, EB virus, human papillomavirus, human T-lymphotropic virus, human herpesvirus 8 and human immunodeficiency virus.

Also, the polysaccharide constituting the hydrophobized polysaccharide is preferably a pullulan or a mannan.

Also, a hydrophobic group of the hydrophobized polysaccharide is preferably a cholesterol.

Also, the hydrophobized polysaccharide is preferably non-ionic.

Also, the particle size after complexing of the hydrophobized polysaccharide and the synthetic long peptide is preferably 180 nm or less. Also, the particle size after complexing of the hydrophobized polysaccharide and the synthetic long peptide is more preferably 100 nm or less.

Also, the immunopotentiating agent is preferably an agonist of a Toll-like receptor. At this time, the agonist of the Toll-like receptor is preferably a poly-IC RNA or a CpG oligo DNA.

Also, the immunopotentiating agent is preferably a chemotherapeutic agent which activates antigen-presenting cells. At this time, the chemotherapeutic agent activating the antigen-presenting cells is preferably a taxane-based compound or an anthracycline-based compound.

Also, the immunopotentiating agent is preferably an agent having the function of preventing antigen-presenting cells from acquiring immunosuppressive activity. At this time, the agent having the function of preventing antigen-presenting cells from acquiring immunosuppressive activity is preferably a JAK/STAT inhibitor or an IDO inhibitor.

In the present invention, the long peptide is a polypeptide characterized in that at least two or more T-cell recognition epitopes are included in a tumor-specific antigenic protein and/or pathogen-derived antigenic protein. The T-cell recognition epitope may be any epitope included in the tumor-specific antigenic protein or in the pathogen-derived antigenic protein, and can be selected from: T-cell recognition epitopes included in tumor-specific antigenic proteins such as the MAGE family molecules like MAGE-A1, MAGE-A2, MAGE-A3, MAGE-A4, MAGE-A5, MAGE-A6, MAGE-A8, MAGE-A9, MAGE-A10, MAGE-A11, MAGE-A12, MAGE-B1 and MAGE-B2; NY-ESO-1 molecules; LAGE; the SAGE family molecules; the XAGE family molecules; the SPANX family molecules; HER2; WT1; hTERT; RHAMM; and survivin; and T-cell recognition epitopes included in pathogen-derived antigenic proteins such as hepatitis B virus (HBV), hepatitis C virus (HCV), EB virus, human papillomavirus (other than type 16 and type 18, types 31, 33, 35, 39, 45, 51, 52, 56, 58, 59, 68, 69, 73 and 82 can be selected), human T-lymphotropic virus (HTLV-1), human herpesvirus 8 and human immunodeficiency virus. The T-cell recognition epitope includes a CTL epitope recognized by killer T cells and a Th epitope recognized by helper T cells. Although it is preferable that the long peptide of the present invention simultaneously contains at least one of each of the CTL epitope and the Th epitope, a long peptide comprising the CTL epitope and a long peptide comprising the Th epitope can be used alone or in combination.

In case the length of the long peptide is 22 amino acids or less, since they cause immune tolerance by directly binding to MHC molecules, it is not possible to achieve the effects of the present invention and thus they are undesirable. In case the length is 120 amino acids or longer, since the effects of the invention cannot be sufficiently achieved, they are also undesirable. Specifically, 23-120 amino acids are preferable, 23-80 amino acids are more preferable, and 23-40 amino acids are even more preferable. Two or more of the long peptides may be combined for use.

The hydrophobized polysaccharide used in the present invention can be manufactured, for example, by the well-known methods described in Non Patent Documents 11-12, Patent Documents 2-4 and the like.

If the sugar residue is a glycosidically-bound polymer, the polysaccharide in the hydrophobized polysaccharide is not particularly limited. As the sugar residue constituting the polysaccharide, for example, residues derived from a saccharide such as a monosaccharide like glucose, mannose, galactose and fucose, or a disaccharide or an oligosaccharide can be used. The sugar residue may be 1,2-, 1,3-, 1,4- or 1,6-glycosidically-bound, and the bonds may be either α- or β-bonds. In addition, the polysaccharide may be either linear or branched. As the sugar residue, glucose residues are preferable; as the polysaccharide, natural or synthetic pullulan, dextran, amylose, amylopectin or mannan can be used, preferably mannan, pullulan or the like. A polysaccharide having an average molecular weight of 50,000-150,000 can be used.

As the hydrophobic group(s), for example, preferably 1 to 5 single or double-stranded alkyl groups or sterol residues are introduced per 100 monosaccharides (5% or less in weight ratio), and more preferably 1 to 3 groups are introduced per 100 monosaccharides (3% or less in weight ratio). Note that the hydrophobic group(s) is (are) not limited to the above-described groups, and groups having a high encapsulation ratio can suitably be selected according to the molecular weight and the isoelectric point of the antigen to be encapsulated. Sterol residues may include, for example, residues of cholesterol, stigmasterol, β-sitosterol, lanosterol, ergosterol or the like; preferably a cholesterol residue is used. In addition, as the alkyl group, an alkyl group having preferably less than 20 carbon atoms, more preferably 10-18 carbon atoms can be used, and it may be either linear or branched.

As the hydrophobized polysaccharide, for example, a polysaccharide is preferable in which a primary hydroxyl group having 1-5 sugar units per 100 sugar units constituting the polysaccharide is represented by the following formula (I): —O—(CH₂)ₘCONH(CH₂)ₙNH—CO—O—R (I) (wherein R is an alkyl group or a sterol residue; m is 0 or 1; n is any positive integer). Here, the above-described groups can be preferably used as the alkyl group or the sterol residue, wherein n is preferably 1 to 8.

Note that the hydrophobized polysaccharide may be a polysaccharide bound via the linker described in Patent Document 4.

Also, the hydrophobized polysaccharide is preferably non-ionic, and the zeta potential under physiological conditions after complexing with the long peptide is preferably −2.0 mV to +2.0 mV. The particle size under physiological conditions after complexing with the long peptide is preferably less than 180 nm, more preferably 100 nm or less.

The complex of the long peptide and the hydrophobized polysaccharide can be purified by various chromatography techniques after the long peptide and aggregated microparticles of the hydrophobized polysaccharide are mixed at room temperature or at a low temperature (Non Patent Document 13). Although the resulting complex of the long peptide and the hydrophobized polysaccharide can be directly used as the vaccine preparation of the present invention, it is also possible to perform a process such as sterilization according to a conventional method, as required.

Any agent having the function of enhancing the activity of antigen-presenting cells may be the immunopotentiating agent; more specifically, Toll-like receptor agonists, other antigen-presenting cell-stimulating agents, and agents having the function of inhibiting antigen-presenting cells from acquiring immunosuppressive activity can be selected. The Toll-like receptor agonist can be selected from an inactivated bacterial cell and a bacterial extract, a nucleic acid, a lipopolysaccharide, a lipopeptide, a synthetic low-molecular compound and the like; preferably, CpG oligo DNA, poly-IC RNA, monophosphoryl lipid, lipopeptide, or the like is used. As the antigen-presenting cell-stimulating agent, a taxane-based agent or an anthracycline-based agent can be used. As the agent having the function of inhibiting antigen-presenting cells from acquiring immunosuppressive activity, it can be selected from a JAK/STAT inhibitor, an indole deoxygenase (IDO) inhibitor, a tryptophane deoxygenase (TDO) inhibitor and the like. Within such inhibitors are included other compounds having antagonistic activity against the factor, as well as neutralizing antibodies for the factor, small interfering RNA (siRNA) and antisense DNA.

Although the vaccine for cancer treatment of the present invention can be administered according to any method, it is preferably administered via a suitable parenteral route, for example, via intravenous, interperitoneal, subcutaneous, intracutaneous, intra-adipose and intramammary routes; via inhalation; via intramuscular injection; or according to a method via a mucosal route in the form of nasal drops or the like; etc.

The vaccine for cancer treatment of the present invention can generally be a preparation in a form suitable for subcutaneous, intravenous and intramuscular parenteral administration, as a kit in which the active ingredients, i.e. the complex of the synthetic long peptide and the hydrophobized polysaccharide and the immunopotentiating agent component, are separately prepared. The dosage of the vaccine preparation for cancer treatment of the present invention required for inducing the intended immunity can be suitably determined: as a normal dosage, e.g. an amount of about 0.1 mg to 5 mg/dose of synthetic long peptide is administered as a reference. It is important that the active ingredients of both preparations coexist substantially simultaneously at the site of administration, and therefore it desired that both preparations are administered substantially at the same time or successively. It is appropriate to carry out the administration 2 to 20 times. Although the administration interval is selected from 1 to 12 weeks, longer intervals between administrations may be selected in a later phase of the treatment.

### Effects of the Invention

According to the present invention, a vaccine for cancer treatment having remarkably high therapeutic effects on cancer can be provided.

### BRIEF DESCRIPTION OF THE DRAWINGS

FIG. 1 A complex (CHP-MAGE-A4, p264:40 complex) was prepared of a synthetic long peptide having 40 amino acids, derived from an amino acid sequence of tumor-specific protein MAGE-A4 (MAGE-A4, p264:40), and CHP of one type of hydrophobized polysaccharide (wherein 1.7 cholesteryl groups were introduced into a pullulan per 100 monosaccharides. Average molecular weight: 100,000). The MAGE-A4 p264:40 or the CHP-MAGE-A4 p264:40 complex was subcutaneously administered to mice simultaneously with CpG oligo DNA as an immunopotentiating agent (twice, at one week intervals). One week after the final administration, spleen cells were collected to measure, using an intracellular cytokine staining method, percentages of CD8-positive killer T cells (FIG. 1A) or CD4-positive helper T cells (FIG. 1B) specific to epitopes contained in the synthetic long peptide.

FIG. 2 An experiment similar to FIG. 1 was performed using a poly-IC RNA as the immunopotentiating agent. The black bars and the white bar respectively represent percentages of antigen-specific CD8-positive killer T cells and antigen-specific CD4-positive helper T cells.

FIG. 3 An experiment similar to FIG. 2 was performed using the synthetic long peptide mERK2 p121:37 of 37 aa, which was derived from an amino acid sequence of a mutated ERK2 (mERK2) that is a mouse tumor antigen, as the synthetic long peptide. The values are percentages of antigen-specific CD8-positive killer T cells.

FIG. 4 An experiment similar to FIG. 1 was performed using, as the hydrophobized polysaccharide, CHP, CHP—NH2 (wherein 2 cholesteryl groups and 13 amino groups were introduced into a pullulan per 100 monosaccharides. Average molecular weight: 100,000), CHP—COOH (wherein 1.2 cholesteryl groups and 27 carboxyl groups were introduced into a pullulan per 100 monosaccharides. Average molecular weight: 100,000) and CHESG (wherein 0.8 cholesteryl groups were introduced into a glycogen per 100 monosaccharides. Average molecular weight: 2,100,000), each of them was complexed with the synthetic long peptide MAGE-A4 p264:40. The particle sizes of each hydrophobized polysaccharide-synthetic long peptide complex and the zeta potentials are shown in Table 1.

FIG. 5 An experiment similar to FIG. 1 was performed using synthetic long peptides of 40 aa, 80 aa or 120 aa derived from the amino acid sequence of MAGE-A4 (MAGE-A4 p264:40, MAGE-A4 p264:80, MAGE-A4 p264:120). The p264:80 and p264:120 respectively comprise 2 or 3 repeated sequences of p264:40.

FIG. 6 An experiment similar to FIG. 1 was performed using synthetic long peptides of 37 aa or 74 aa derived from the amino acid sequence of mERK2 (mERK2 p121:37, mERK2 p121:74). The p121:74 comprises 2 repeated sequences of p121:37.

FIG. 7 The MAGE-A4 p264:40 or the CHP-MAGE-A4 p264:40 complex was subcutaneously administered to BALB/c mice simultaneously with CpG oligo DNA. Seven days after the administration, mouse colon cancer cells CT 26 into which the MAGE-A4 gene was stably introduced were subcutaneously implanted. The subsequent changes of tumor areas in each individual are shown in the graphs. Both the administration group of MAGE-A4 p264:40/CpG oligo DNA and the administration group of CHP-MAGE-A4 p264:40 complex/CpG oligo DNA showed significant inhibition (p<0.0001) of tumor growth as compared to an administration group of phosphate buffered saline (PBS), but the CHP-MAGE-A4 p264:40 complex/CpG oligo DNA administration group showed more remarkable growth inhibition effects.

FIGS. 8 The mERK2 p121:37 or the CHP-mERK2 p121:37 complex was subcutaneously administered to BALB/c mice simultaneously with CpG oligo DNA. Seven days after the administration, mouse fibrosarcoma cells CMS5a which express mERK2 protein were subcutaneously implanted. The subsequent changes of tumor growth in each individual are shown in the graphs. Only the CHP-mERK2 p121:37 complex/CpG oligo DNA administration group showed significant inhibition (p<0.03) of tumor growth as compared to the PBS administration group.

### MODES FOR CARRYING OUT THE INVENTION

Next, embodiments of the present invention will be explained with reference to the Figures and tables, but the technical scope of the present invention is not limited by these embodiments and can be carried out in various configurations without changing the gist of the invention. In addition, the technical scope of the present invention extends to the scope of equivalents.

### <Materials and Methods>

(1) Materials

BALB/c mice (females, 5 weeks old) were purchased from Japan SLC, Inc., and raised in the Animal Center of the Faculty of Medicine of Mie University. After one week of acclimation, they were used for the experiments. The experiment protocol using the mice received approval from the Ethics Committee of the Faculty of Medicine of Mie University. Synthetic long peptides were purchased from GenScript Inc., Scrum Inc., and Biologica Co. The sequences of the synthetic long peptides are as follows: MAGE-A4 p264:40 (amino acid sequence (SEQ ID NO: 1): GSNPARYEFLWGPRALAETSYVKVLEHVVRVNARVRIAYP, wherein the sequence of 9 amino acids from the 2th S to the 10th L (SEQ ID NO: 2: SNPARYEFL) is included as the killer T cell epitope sequence, and the sequence of 16 amino acids from the 22th V to the 37th (SEQ ID NO: 3: VKVLEHVVRVNARVRI) is included as the helper T cell epitope). MAGE-A4 p264:80 (amino acid sequence (SEQ ID NO: 4): GSNPARYEFLWGPRALAETSYVKVLEHVVRVNARVRIAYPGSNPARYEFLWGPRALAETSYV KVLEHVVRVNARVRIAYP, two repeated sequences of p264:40). MAGE-A4 p264:120 (amino acid sequence (SEQ ID NO: 5): GSNPARYEFLWGPRALAETSYVKVLEHVVRVNARVRIAYPGSNPARYEFLWGPRALAETSYV KVLEHVVRVNARVRIAYPGSNPARYEFLWGPRALAETSYVKVLEHVVRVNARVRIAYP, three repeated sequences of p264:40). mERK2 p121:37 (amino acid sequence (SEQ ID NO: 6): NDHIAYFLYQILRGLQYIHSANVLHRDLKPSNLLLNT, wherein the sequence of 9 amino acids from the 16th Q to the 24th L (SEQ ID NO: 7: QYIHSANVL) is included as the killer T cell epitope sequence, and the sequence of 17 amino acids from the 13th R to the 29th K (SEQ ID NO: 8: RGLQYIHSANVLHRDLK) is included as the helper T cell epitope sequence). mERK2 p121:74 (amino acid sequence (SEQ ID NO: 9): NDHIAYFLYQILRGLQYIHSANVLHRDLKPSNLLLNTNDHIAYFLYQILRGLQYIHSANVLH RDLKPSNLLLNT, two repeated sequences of p121:37). Synthetic peptides were purchased from Sigma-Genosys. The amino acid sequences of the peptides are as follows: MAGE-A4 p265 (amino acid sequence (SEQ ID NO: 2): SNPARYEFL), mERK2 9m (amino acid sequence (SEQ ID NO: 7): QYIHSANVL). CHP was purchased from NOF (Corp.). With respect to the synthesis of CHP—NH2, under a nitrogen atmosphere CHP and 1,1′-carbonyldiimidazole were reacted using DMSO as a solvent, to which ethylenediamine was then added to react with it. The reaction liquid was dialyzed and purified, then lyophilized to obtain CHP—NH2. With respect to the synthesis of CHP—COOH, under a nitrogen atmosphere CHP and a succinic anhydride were reacted using DMSO as a solvent and N,N-dimethyl-4-aminopyridine as a catalyst. After the reaction, the product was re-precipitated and dialyzed and then lyophilized to obtain CHP—COOH. With respect to the synthesis of CHESG, under a nitrogen atmosphere an enzyme-synthesized glycogen and cholesteryl isocyanate were reacted using DMSO as a solvent and dibutyltin dilaurate as a catalyst. After the reaction, the product was re-precipitated and dialyzed and then lyophilized to obtain CHESG. As the immunopotentiating agent, poly-IC RNA was purchased from Oncovir, Inc. CpG oligo DNA was purchased from Hokkaido System Science Co., Ltd. Imiquimod was purchased from Sigma-Aldrich Co. FITC-labeled anti-CD4 monoclonal antibody (clone RM4-5), PerCP-Cy5.5-labeled anti-CD8 monoclonal antibody (clone 53-6.7), APC-labeled anti-IFNγ antibody (clone XMG1.2) and PE-labeled anti-IL-2 antibody (clone JES6-5H4) were purchased from eBioscience, Inc., or BD Biosciences Company.

(2) Preparation of the Complex of the Hydrophobized Polysaccharide and the Synthetic Long Peptide

10 mg/mL of synthetic long peptide was dissolved in dimethylsulfoxide (DMSO). 10 mg/mL of hydrophobized polysaccharide was dissolved in phosphate buffered saline (PBS) containing 6 M of urea. 20 mL (200 mg) of the hydrophobized polysaccharide solution was added to 1 mL (10 mg) of the synthetic long peptide solution, and left in a dark room at room temperature overnight. It was transferred to a dialysis device (molecular weight cut-off: 3500, Thermo Fisher Scientific K.K.), and dialyzed at room temperature for 2 hours to overnight against PBS containing 0.6 M of urea as the dialysate fluid at a volume ratio of more than a hundredfold. Subsequently, it was dialyzed against PBS containing 0.06 M of urea as the dialysate fluid at room temperature for 2 hours to overnight at a volume ratio of more than a hundredfold. It was dialyzed again against PBS as the dialysate fluid at room temperature for 2 hours to overnight at a volume ratio of more than a hundredfold. The non-dialyzable fluid was collected, and UV absorption (280 nm) was measured to determine the final concentration of the synthetic long peptide. The particle size of the hydrophobized polysaccharide-synthetic long peptide complex was measured using a dynamic light scattering photometer (Zetasizer, Malvern Instruments Ltd.), and the Z-Average size was utilized. The zeta potential was measured using a zeta potential measuring device (SZ-100, HORIBA, Ltd.).

(3) Administration Method

The hydrophobized polysaccharide-synthetic long peptide complex and the immunopotentiating agent were simultaneously administered to mice. For administration, they were subcutaneously injected into the backs of the mice twice at one week intervals. For one dose of the hydrophobized polysaccharide-synthetic long peptide complex, a dosage corresponding to 0.1 mg of the synthetic long peptide complex was administered. Either the CpG oligo DNA or the poly-IC RNA as the immunopotentiating agent was subcutaneously injected at a dose of 0.05 mg proximal to the administration site of the hydrophobized polysaccharide-synthetic long peptide.

(4) Isolation and Purification of Mouse Immunocytes

One week after the final administration, spleen cells were isolated from the treated mice in the following manner. The spleen was isolated from the mouse and washed in RPMI1640 medium to remove blood. The spleen was ground using a slide glass, and then the free cells were collected in RPMI1640 medium. After centrifugation (400×g, 5 min., 4° C.), the supernatant was removed, to which 2 mL of ACK solution was added to treat the cells for 1 minute. 18 mL of RPMI1640 medium was added and centrifugation (400×g, 5 min., 4° C.) was carried out. The supernatant was removed, and the cells were suspended in an adequate amount of RPMI1640 medium. After the number of the cells was counted, the cells were suspended in RPMI1640 medium containing 10% fetal bovine serum (FBS) so that the cell concentration was 1×10⁷ cells/mL.

(5) Intracellular Cytokine Staining

The isolated spleen cells were added to a 24-well culture plate (Nunc Co., Ltd.) at 5×10⁶/0.5 mL per one well. As the peptide for re-stimulation, 10 μM of MAGE-A4 p264, MAGE-A4 p265 or mERK2 9m was added and culturing was performed at 37° C. for 6 hours under 5% CO₂. Subsequently, BD GoldiPlug™ (BD Biosciences Company) 10-fold-diluted with RPMI1640 medium containing 10% FBS was added at 50 μL/well, and culturing was performed at 37° C. for 6 hours under 5% CO₂. The cells were collected and transferred to a 96 well round-bottom microplate (Nunc Co., Ltd.). After the supernatant was removed by centrifugation (1200 rpm, 1 min., 4° C.), the cells were suspended in a staining buffer (PBS containing 0.5% bovine serum albumin) at 50 μL/well. A manufacturer-recommended amount of FITC-labeled anti-CD8 monoclonal antibody or FITC-labeled anti-CD4 monoclonal antibody was added, mixed, and then it was left in a dark room at 4° C. for 15 minutes. After the cells were washed twice with 200 μL of staining buffer, 100 μL of BD Cytofix/Cytoperm™ buffer (BD Biosciences Company) was added and mixed very gently. It was left in a dark room at room temperature for 20 minutes, and then washed twice with 100 μL of BD Perm/Wash™ buffer (BD Biosciences Company). 50 μl of BD Perm/Wash™ buffer containing various anti-cytokine antibodies was added to the cells, which were gently suspended and then left in a dark room at room temperature for 15 minutes. The cells were washed twice with 100 μL of BD Perm/Wash™ buffer, then re-suspended in 200 μl of staining buffer, and transferred to a round-bottom polystyrene tube (BD Biosciences Company).

(6) Flow Cytometry Analysis

The cells were analyzed with a flow cytometer (FACS Canto II, BD Biosciences Company) using the associated analysis software (FACSDiva).

(7) Tumor Rejection Test

A mouse colon cancer cell CT26 cell line into which the MAGE-A4 gene was stably introduced or a mouse fibrosarcoma CMS5a cell line which expresses mERK2 was subcutaneously implanted in the following manner. The CT26 cell line or CMS5a cell line, which had been cultured in a T75 flask (Nunc Co., Ltd.), was washed with PBS; then the cells were removed with PBS containing 0.5% trypsin and collected in RPMI1640 medium containing 10% FBS. After centrifugation (400×g, 5 min., 4° C.), the supernatant was removed, washed twice with RPMI1640 medium, then re-suspended in RPMI1640 medium at a concentration of 1×10⁶ cells/100 μL, and subcutaneously implanted into BALB/c mice in an amount of 100 μL/individual. Subsequently, their tumor areas were measured over time.

(8) Statistical Analysis

A comparison of the data in the tumor rejection test was performed according to Student's t-test using MS Excel (Microsoft).

<Test Results>

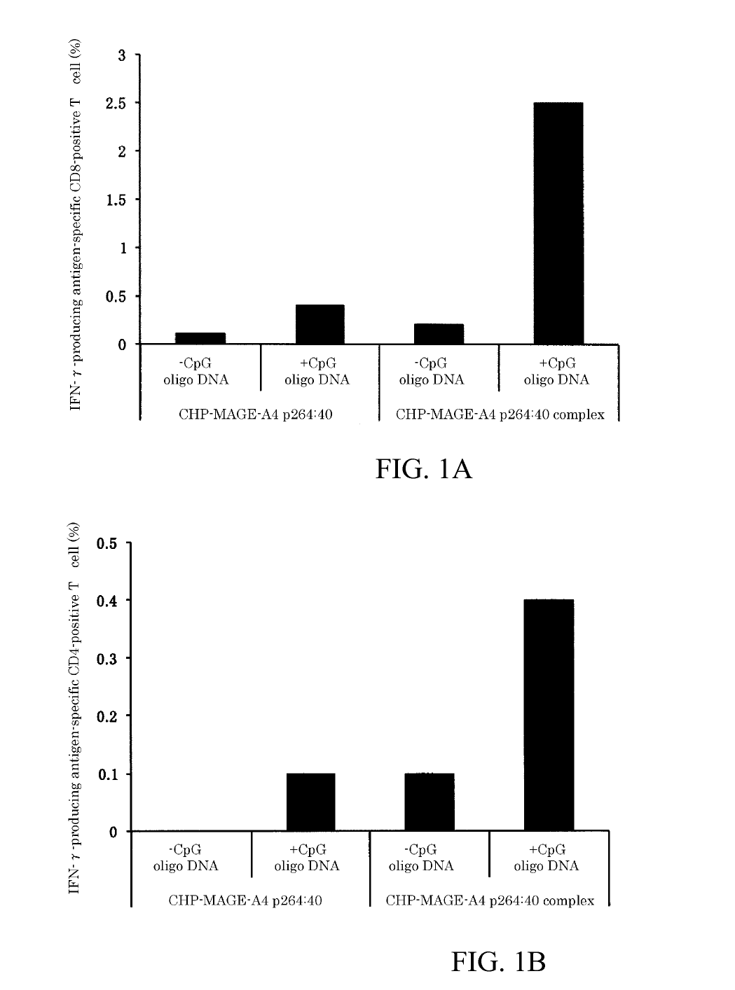

The graphs of FIG. 1 show the clear induction of killer T cells and helper T cells specific for the synthetic long peptide serving as the antigens by co-administering the immunopotentiating agent with the hydrophobized polysaccharide complexed with the synthetic long peptide. When the synthetic long peptide of 40 amino acids (aa) derived from the tumor-specific protein MAGE-A4 (MAGE-A4 p264:40) is administered alone to mice, killer T cells and helper T cells specific to said peptide are not induced. When the CpG oligo DNA (Toll-like receptor 9 agonist) is co-administered as the immunopotentiating agent with the peptide, the intended T cells are not induced. However, when the peptide is co-administered with the CpG oligo DNA after having been complexed with CHP, which is one type of hydrophobized polysaccharide, induction of antigen-specific killer T cells and helper T cells can be clearly observed. When only the CHP-MAGE-A4 p264:40 complex is administered, such a T-cell induction does not occur. From this, it has been found that a long peptide vaccine can enjoy the maximum enhancing effects of the CpG oligo DNA, which is an immunopotentiating agent, only after being complexed with the hydrophobized polysaccharide CHP.

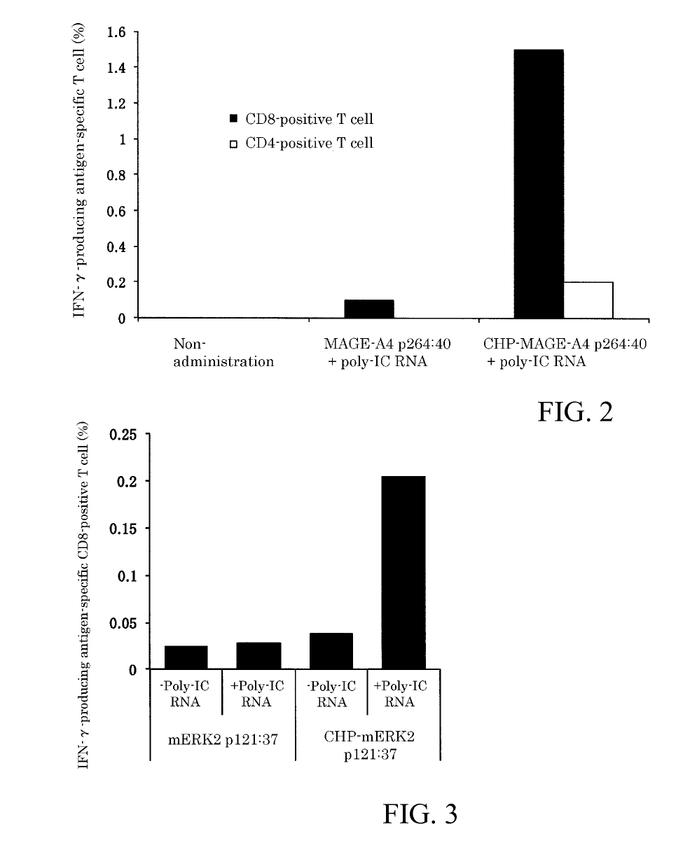

A test similar to FIG. 1 was performed using poly-IC RNA (Toll-like receptor 3 agonist) as the immunopotentiating agent instead of the CpG oligo DNA. As anticipated, the effects of the immunopotentiating agent were more remarkable with the CHP-synthetic long peptide complex than with the synthetic long peptide alone (FIG. 2). These results suggest that as long as the immunopotentiating agent, which enhances immune induction by administration of CHP-synthetic long peptide complexes, can activate antigen-presenting cells, such as a Toll-like receptor agonist, any of them can be broadly used.

Results similar to FIG. 2 were reproduced even when the synthetic long peptide was changed from the MAGE-A4 protein-derived p264:40 to the mERK2 protein-derived p121:37 (37 aa) (FIG. 3). These results indicate that clear immune induction by co-administration of the CHP-synthetic long peptide complex and the immunopotentiating agent is not limited to synthetic long peptides having particular sequences.

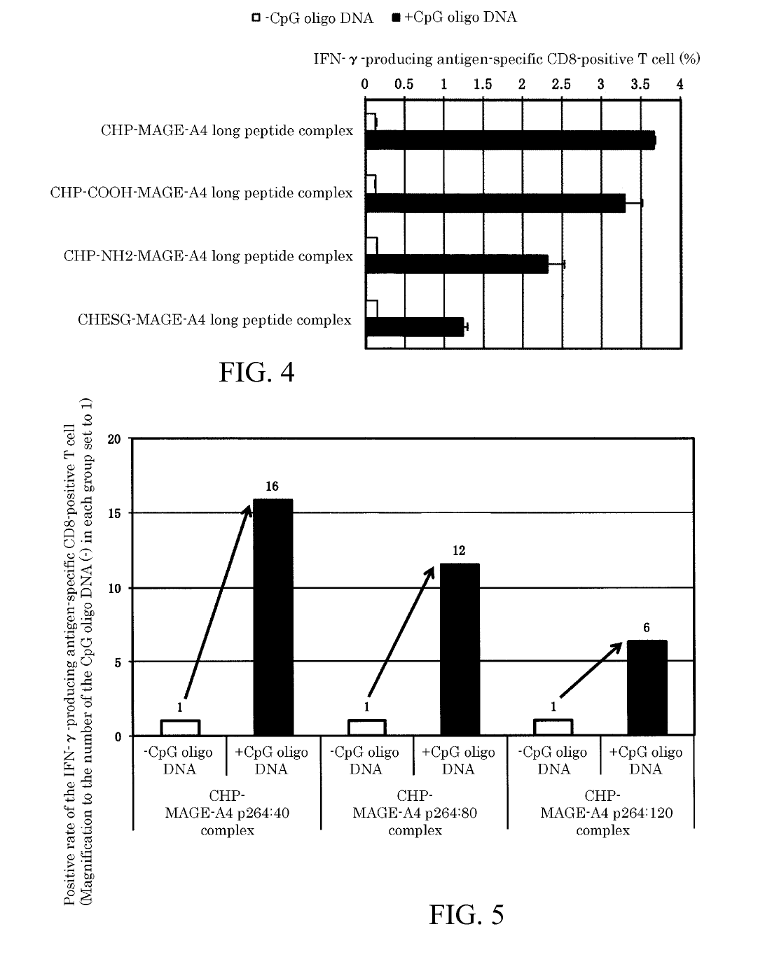

In order to investigate the effects of the size and charge of the hydrophobized polysaccharide on the vaccine performance, tests similar to FIG. 1 were performed except that CHP was replaced with CHP—NH2, CHP—COOH or CHESG (FIG. 4). Compounds in which the non-ionic CHP is rendered ionic by chemical modification are CHP—NH2 and CHP—COOH (Table 1). However, after CHP—COOH is complexed with the synthetic long peptide, its zeta potential is approximately zero and it is actually non-ionic. CHESG is obtained by replacing the sugar chain moiety with glycogen to enlarge the particle size (Table 1). As a result of the tests, the enhancing effects by the CpG oligo DNA were clearly attenuated in the administration groups of the long peptide vaccine complexed with CHP—NH2 and CHESG. In the administration group of the long peptide vaccine complexed with CHP—COOH, the enhancing effects by the CpG oligo DNA were not remarkably attenuated. From this, it became clear that the hydrophobized polysaccharide used in the present invention advantageously satisfies the conditions that it is non-ionic after complexing with the synthetic long peptide, and the particle size is less than about 180 nm, preferably less than about 100 nm. In conventional vaccine antigen delivery systems that use liposomes, it has been reported that the larger the size is and the higher the charge is, the better the immunological induction by the vaccine is (Non Patent Documents 14-16). However, in the case of the hydrophobized polysaccharides of the present invention, it is understood that the smaller the size is after complexing with the long peptide, the more effective the non-ionicity is after complexation, and consequently it has become clear it is completely different from the case of the conventional liposome.

TABLE 1

| | | Before complexing | | After complexing with the | |
|---|---|---|---|---|---|
| | | with the synthetic | | synthetic long peptide | |
| | Average | long peptide | | (MAGE-A4 p264:40) | |
| Hydrophobized | molecular | Particle | Zeta | Particle | Zeta |
| polysaccharide | weight | size, nm | potential, mV | size, nm | potential, mV |
| CHP | 100,000 | 31 | N/A | 55 | 0.3 |
| CHP-NH₂ | 100,000 | 39 | 6.9 | 86 | 16.1 |
| CHP-COOH | 100,000 | 30 | −14.7 | 44 | −1.1 |
| CHESG | 2,100,000 | 115 | N/A | 183 | −1.9 |

Tests similar to FIG. 1 were performed except that the length of the long peptide was changed from 40 aa to 80 aa or 120 aa (FIG. 5). The sequence of the long peptide of 80 aa or 120 aa is respectively two or three repeated sequences of the 40 aa long peptide. Although complexes of the long peptides of the respective lengths with CHP induced the intended killer T cell response, the longer the amino acid length of the long peptide was, the weaker the enhancing effects by the CpG oligo DNA became. From this, it became clear that the length of the long peptide used in the present invention is 120 aa or less, preferably 80 aa or less, more preferably 40 aa or less.

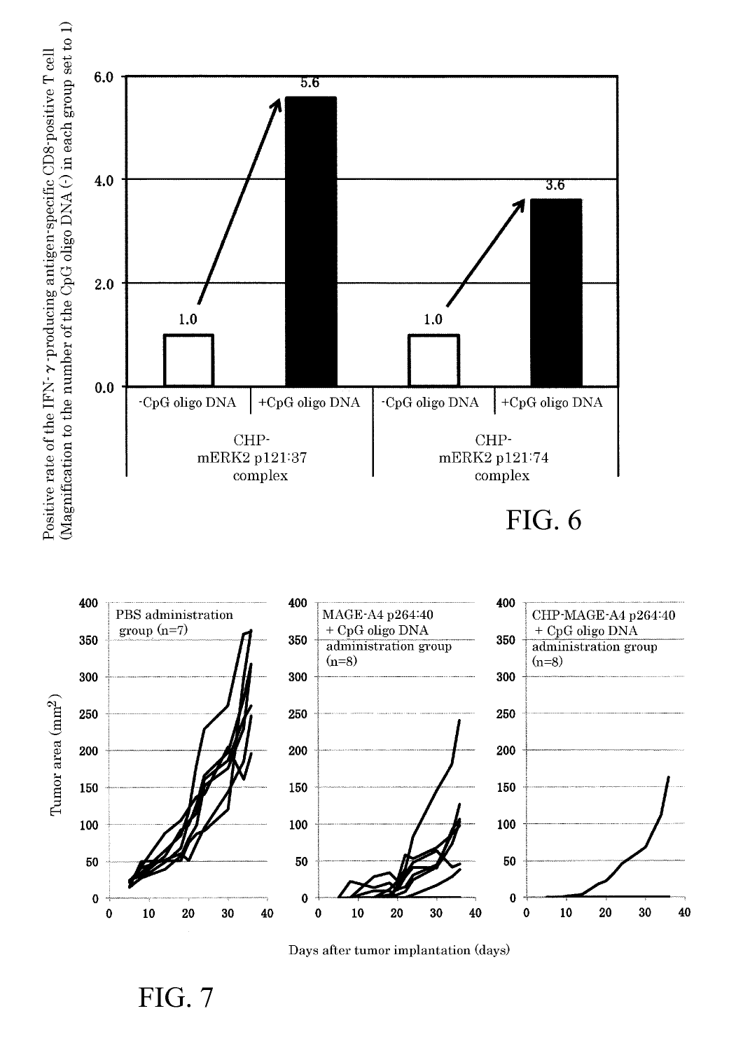

Tests similar to FIG. 1 were performed except that the long peptide was changed from the MAGE-A4 protein-derived peptide to the mERK2 protein-derived peptide (FIG. 6). Similar to FIG. 5, the longer the amino acid length of the long peptide was, the weaker the enhancing effects by the CpG oligo DNA became.

In a cancer-bearing mouse model in which mouse colon cancer cells CT 26 expressing the MAGE-A4 protein were implanted, significant tumor growth inhibitory effects were observed with the co-administration group of the CHP-MAGE-A4 p264:40 complex and the CpG oligo DNA as compared to the PBS administration group (p<0.0001), and the tumor growth inhibitory effects were significantly superior even as compared to the co-administration group of the MAGE-A4 p264:40 and the CpG oligo DNA (p<0.05) (FIG. 7). The results are in good agreement with the ability of each vaccine shown in FIG. 1 to induce antigen-specific T cells.

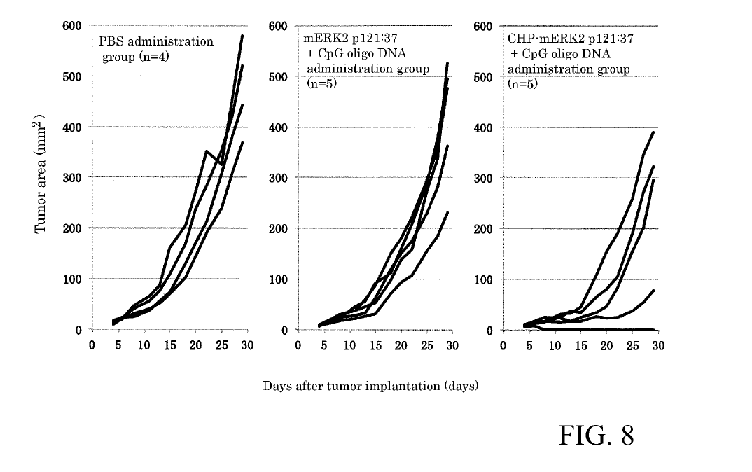

Also in a cancer-bearing mouse model in which mouse fibrosarcoma cells CMS5a expressing the mERK2 protein were implanted, significant tumor growth inhibitory effects were observed with the co-administration group of the CHP-mERK2 p121:37 complex and the CpG oligo DNA as compared to the PBS administration group (p<0.03) (FIG. 8). On the other hand, significant tumor growth inhibitory effects were not observed in the co-administration group of mERK2 p121:37 and the CpG oligo DNA.

These results show that, with the administration of the synthetic long peptide alone, with the co-administration of the synthetic long peptide and the immunopotentiating agent, and with the administration of the hydrophobized polysaccharide-synthetic long peptide complex alone, induction of antigen-specific killer T cells and helper T cells was insufficient; although this shows that therapeutic effects as vaccines for cancer treatment can not be expected, in case of co-administration of the hydrophobized polysaccharide-synthetic long peptide complex and the immunopotentiating agent, it has been clearly shown that the vaccine can enjoy the maximum effects of the immunopotentiating agent, and can achieve the manifestation of therapeutic effects on tumors with activation of antigen-specific T cells. Also, the physicochemical properties of the hydrophobized polysaccharide and the peptide length of the long peptide required for the present invention to exhibit the maximum effects have been elucidated.

According to the present exemplary embodiments, vaccines for cancer treatment having extremely high therapeutic effects on cancers could be provided.

1. A vaccine preparation for treating cancer comprising;
a complex of a hydrophobized polysaccharide and at least one synthetic long peptide derived from a tumor-specific antigenic protein and/or a pathogen-derived antigenic protein, the at least one synthetic long peptide containing at least one CD8 positive, cytotoxic T-cell recognition epitope and at least one CD4 positive, helper T-cell recognition epitope; and
at least one immunopotentiating agent.

2. The vaccine preparation according to claim 1, wherein the at least one synthetic long peptide is a sequence of 23-120 amino acids, wherein both of the at least one CD8 positive, cytotoxic T-cell recognition epitope and the at least one CD4 positive, helper T-cell recognition epitope are contained in the sequence.

3. (canceled)

4. The vaccine preparation according to claim 1, wherein the at least one synthetic long peptide is a sequence of 23-80 amino acids, wherein both of the at least one CD8 positive, cytotoxic T-cell recognition epitope and the at least one CD4 positive, helper T-cell recognition epitope are contained in the sequence.

5. The vaccine preparation according to claim 1, wherein the at least one synthetic long peptide is a sequence of 23-40 amino acids, wherein both of the at least one CD8 positive, cytotoxic T-cell recognition epitope and the at least one CD4 positive, helper T-cell recognition epitope are contained in the sequence.

6. The vaccine preparation according to claim 1, wherein the at least one CD8 positive, cytotoxic T-cell recognition epitope is a part of an amino acid sequence of the tumor-specific antigenic protein.

7. The vaccine preparation according to claim 1, wherein the at least one CD4 positive, helper T-cell recognition epitope is a part of an amino acid sequence of the tumor-specific antigenic protein.

8. The vaccine preparation according to claim 1, wherein the tumor-specific antigenic protein is selected from the group consisting of the MAGE family, NY-ESO-1, the SPANX family, HER2, WT1, hTERT, RHAMM and survivin.

9. The vaccine preparation according to claim 1, wherein the at least one CD8-positive helper T-cell recognition epitope is a part of an amino acid sequence of the pathogen-derived antigenic protein.

10. The vaccine preparation according to claim 1, wherein the at least one CD4 positive, helper T-cell recognition epitope is a part of an amino acid sequence of the pathogen-derived antigenic protein.

11. The vaccine preparation according to claim 1, wherein the pathogen-derived antigenic protein is derived from a virus selected from the group consisting of hepatitis B virus, hepatitis C virus, EB virus, human papillomavirus, human T-lymphotropic virus, human herpesvirus 8 and human immunodeficiency virus.

12. The vaccine preparation according to claim 1, wherein the hydrophobized polysaccharide comprises a pullulan or a mannan.

13. The vaccine preparation according to claim 1, wherein the hydrophobized polysaccharide comprises at least one cholesterol group.

14. The vaccine preparation according to claim 1, wherein the hydrophobized polysaccharide is non-ionic.

15. The vaccine preparation according to claim 1, wherein the complex of the hydrophobized polysaccharide and the at least one synthetic long peptide has a particle size of 180 nm or less.

16. The vaccine preparation according to claim 1, wherein the complex of the hydrophobized polysaccharide and the at least one synthetic long peptide has a particle size of 100 nm or less.

17. The vaccine preparation according to claim 16, wherein the at least one immunopotentiating agent is an agonist of a Toll-like receptor.

18. The vaccine preparation according to claim 17, wherein the agonist of the Toll-like receptor is a poly-IC RNA or a CpG oligo DNA.

19. The vaccine preparation according to claim 1, wherein the at least one immunopotentiating agent is a chemotherapeutic agent which activates antigen-presenting cells.

20. The vaccine preparation according to claim 19, wherein the chemotherapeutic agent which activates antigen-presenting cells is a taxane-based compound or an anthracycline-based compound.

21. The vaccine preparation according to claim 1, wherein the at least one immunopotentiating agent is an agent that prevents antigen-presenting cells from acquiring immunosuppressive activity.

22. The vaccine preparation according to claim 21, wherein the agent is a JAK/STAT inhibitor or an IDO inhibitor.

23. The vaccine preparation according to claim 1, wherein:
the at least one synthetic long peptide is derived from MAGE-A4 and
the at least one immunopotentiating agent is a poly-IC RNA and/or a CpG oligo DNA.

24. The vaccine preparation according to claim 23, wherein:
the at least one synthetic long peptide is a sequence of 23-80 amino acids and
both of the at least one CD8 positive, cytotoxic T-cell recognition epitope and the at least one CD4 positive, helper T-cell recognition epitope are contained in the sequence.

25. A method of treating cancer, comprising administering to a patient in need thereof the vaccine preparation of claim 1.

26. A composition of matter comprising:
at least one synthetic long peptide derived from a tumor-specific antigenic protein and/or a pathogen-derived antigenic protein, the at least one synthetic long peptide containing at least one epitope recognized by CD8+ cytotoxic T-cells and at least one epitope recognized by CD4+ helper T-cells; and
a hydrophobized polysaccharide complexed with the at least one synthetic long peptide.

27. The composition of matter of claim 26, wherein the hydrophobized polysaccharide comprises a pullulan or a mannan and further comprises at least one cholesterol group.


SEQUENCE LISTING (9 sequences)

SEQ ID NO: 1 - protein, 40 aa, Human
  MAGE-A4 p26440
GSNPARYEFLWGPRALAETSYVKVLEHVVRVNARVRIAYP

SEQ ID NO: 2 - protein, 9 aa, Human
  MAGE-A4 p265
SNPARYEFL

SEQ ID NO: 3 - protein, 16 aa, Human
VKVLEHVVRVNARVRI

SEQ ID NO: 4 - protein, 80 aa, Artificial
  MAGE-A4 p26480
GSNPARYEFLWGPRALAETSYVKVLEHVVRVNARVRIAYPGSNPARYEFLWGPRALAETSYVKVLEHVVRVNARVRIAYP

SEQ ID NO: 5 - protein, 120 aa, Artificial
  MAGE-A4 p264120
GSNPARYEFLWGPRALAETSYVKVLEHVVRVNARVRIAYPGSNPARYEFLWGPRALAETSYVKVLEHVVRVNARVRIAYPGSNPARYEFLWGPRALAETSYVKVLEHVVRVNARVRIAYP

SEQ ID NO: 6 - protein, 37 aa, Human
  mERK2 p12137
NDHIAYFLYQILRGLQYIHSANVLHRDLKPSNLLLNT

SEQ ID NO: 7 - protein, 9 aa, Human
  9m
QYIHSANVL

SEQ ID NO: 8 - protein, 17 aa, Human
RGLQYIHSANVLHRDLK

SEQ ID NO: 9 - protein, 74 aa, Artificial
  mERK2 p12174
NDHIAYFLYQILRGLQYIHSANVLHRDLKPSNLLLNTNDHIAYFLYQILRGLQYIHSANVLHRDLKPSNLLLNT